(19) United States
(12) Patent Application Publication
(10) Pub. No.: US 2022/0023582 A1
(43) Pub. Date: Jan. 27, 2022

(54) Humidification of an Environment with KELEA Activated Water to Increase the Availability of Health-Promoting KELEA for Those Present Within the Environment

(71) Applicant: William John Martin, South Pasadena, CA (US)

(72) Inventor: William John Martin, South Pasadena, CA (US)

(73) Assignee: MI Hope Inc, South Pasadena, CA (US)

(21) Appl. No.: 16/947,267

(22) Filed: Jul. 26, 2020

(51) Int. Cl. A61M 16/16; A61M 15/02

(52) U.S. Cl. CPC A61M 16/162; A61M 2202/203; A61M 2202/206; A61M 15/02

(57) ABSTRACT

Illnesses result from individuals having insufficient cellular energy for normal functioning and to respond to various adverse events. In addition to receiving cellular energy from the metabolism of food, living creatures are dependent upon an environmental force termed KELEA, an abbreviation for Kinetic Energy Limiting Electrostatic Attraction. This energy drives what the Applicant refers to as the Alternative Cellular Energy (ACE) Pathway. It is expressed as an added dynamic or kinetic quality of the body's fluids. Water from various natural sources has varying levels of acquired KELEA. Various means are available to increase the level of KELEA in water. KELEA activated water has medicinal qualities in the prevention and cure of illnesses attributed to insufficient cellular energy. It is typically used in individual patients being either consumed as drinking water or used topically on the skin as a spray. The present invention describes the novel use of KELEA activated water in a humidifier to increase the overall environmental level of the KELEA activated water vapor in an enclosed environment. This approach is intended to enhance the ACE pathway of those present for; prolonged periods within such an environment. This can provide both incidental and directed benefits for large groups of individuals. This method is especially applicable when using KELEA activated water which is abundantly available from certain locations, including a location specified in this application.

### RELATED PUBLICATIONS BY THE APPLICANT

Martin W J (2014) Stealth Adapted Viruses; Alternative Cellular Energy (ACE) & KELEA Activated Water. Author House, Bloomington, Ind. pp 321 ISBN 978-4969-0496-6

Martin W J (2020) Enhancing the Alternative Cellular Energy (ACE) Pathway with KELEA Activated Water as Therapy for Infectious Diseases. Infectious Disorders—Drug Targets. February 10. doi: 10.2174/1871526520666200211115111.

### PENDING PATENT APPLICATIONS

Method of Enhancing the Alternative Cellular Energy Pathway in Humans and Animals Using Wearable Items That Contain KELEA Activated Water. Submitted Feb. 18, 2019. application Ser. No. 16/278,712

Method of Detection and Measurement of a Life Force Energy, Also Known as KELEA in Liquids & Other Materials. Submitted May 23, 2019. application Ser. No. 16/421,344

Using paper with heightened KELEA to Enhance the alternative cellular energy pathway. Submitted Jul. 10, 2019. application Ser. No. 16/508255

Methods for Treating Vitiligo Based on Enhancing the Alternative Cellular Energy Pathway Using KELEA Activated Water submitted Apr. 25, 2020. Application number

### FIELD OF THE INVENTION

The invention is within the field of cellular energy-based medical therapies.

### BACKGROUND

The human body comprises approximately thirty trillion cells, organized into over two-hundred specialized cell types. Humans are healthy when the cells are functioning normally. To do so, cells require energy. There are three basic levels of cellular energy requirements. The first is sufficient energy to survive. The second is the additional energy required by cells to perform their specialized functions. The third level is the energy required by cells to respond to adverse events. Included in these third level responses are the repair of damaged cells or, if necessary, the regeneration and replacement of dying cells. This third level of energy is also required by the body to combat infections and to eliminate and not to be overly reactive to toxic materials. Repair also includes the process of apoptosis or cell suicide, especially if the cell is destined to become a cancer. A summary of these concepts is provided in the cited two publications, which are incorporated herein by reference.

Diseases can be viewed as an insufficiency of cellular energy at one or more of the three levels of energy requirements. The energy insufficiency can be localized to a particular type of cell or location in the body or can be more generalized. Diseases can have multiple causes.

It had been assumed that human and animal cells could only obtained energy from food metabolism; with the mitochondria being the principal organelle for the food-derived cellular energy. The inventor has proposed with considerable supporting data that humans and animals also relay upon an environmental force, which he has referred to as KELEA, an abbreviation for Kinetic Energy Limiting Electrostatic Attraction. KELEA is seemingly attracted to both positive and negative electrical charges. It provides a repulsive mechanism, which prevents the ultimate fusion and annihilation of electrostatically attached opposing electrical charges. It is proposed that KELEA is present on the isolated electrical charges on dipolar molecules, including water. Increasing the level of KELEA in water, can weaken the attachments between the electrostatically bonded the water molecules. Consequently, the water molecules become more kinetically active. This energy can be transferred to other molecules and, in the opinion of the inventor, comprises the elusive concept of how energy transfer occurs in linked chemical reactions.

Certain dipolar chemicals compounds can both attract and transmit KELEA. This possibly occurs in an oscillating manner in which the added attraction of KELEA leads to a structural change in the compound causing the release of the added KELEA. If water is sufficiently activated, with KELEA its molecular, which now have somewhat more separated electrical charges can undergo a similar oscillation with repetitive KELEA attraction and release. Added regular water can, thereby, become activated. This principle can explain the retention of activity in homeopathic products.

The fluctuating electrical activities of the brain probably act as the major receiver for KELEA. The energy can then be transferred to the body's fluids as a non-food source of cellular energy. Empirically, the effectiveness of the brain as a receiver of KELEA correlates directly with positive emotional attributes and is undermined by feelings of stress, anger, and despair.

It is acknowledged that consuming water from certain specific locations in the world can have remarkable healing benefits for many illnesses. The benefits of water from these specific locations are usually attributed to the differential presence of various biologically active minerals. Healing spars were typically established at these locations, with their medicinal waters being marketed by many of the early pharmacies. With the enormous growth of the bottled water industry, all medical claims have been removed in place of emphasis on taste, purity, and the importance of adequate hydration for optimal health. Good water is also viewed as a source of minerals for the body.

It is broadly argued that the body can also benefit from the consumption of a wide range of naturally occurring products. These include many compounds with stated antioxidant activity. This is, in fact, an indirect indication of the compounds being dipolar. Many herbal products and plant extracts also commonly comprise mixtures of complex dipolar compounds. Other groupings of dipolar molecules include humid and filmic acids, zeolites, mica, mineral oxides, etc.

The inventor has argued that the major beneficial effects of these components is through their ability to attract KELEA and to transfer the energy to the body's fluids. Many of these compounds can also directly attract KELEA into water.

Consistent with this concept are the many reports of being able to enhance the medical benefits of water through various manipulations of the water. The modified water is variously referred to as being activated, structured, micro-clustered, energized, hexagonal, living, functional, etc. These water products are typically marketed for direct consumption as drinking water or for use as a water spray on the skin. They are very occasionally recommended for sublingual use or for inhalation.

The inventor has pioneered the use of wearable waterceutical pouches containing KELEA activated water. These are typically flexible PVC (polyvinyl chloride) containers that are heat sealed. The size and amount of included water has ranged from 60 to >100 ml. Larger containers have been placed between bed sheets for overnight skin contact. These waterceutical pouches rely upon the capacity of the KELEA activated water to act as a perpetual receiver of KELEA, with effective transmission of the energy to the body's fluids. They are most effective when worn directly on the skin. Some individuals have essentially worn the same pouch daily for well over six months, attesting to its continuing benefits. Pouches have been applied to hospitalized patients at the request of the patients' relatives. They have also been successfully used to expedite the recovery of horses from strenuous exercise.

The inventor feels confident that in addition to relieving symptoms, the pouches have consistently improved the emotional wellness of those wearing the pouches. This change is expressed as a renewed sense of optimism, joy, wonderment, and engagement with life and with other individuals. The energy required for these higher levels of brain activity is seemingly not being provided by the calories obtained from the metabolism of food. Rather it appears to be an expression of the ACE pathway. This insight is particularly pertinent if, as believed by the inventor, it also results in the brain is also becoming a more efficient receiver of environmental KELEA. It implies that individuals can move beyond requiring the waterceutical pouches into maintaining a more naturally sustainable ACE pathway.

A further use of the pouches is placing one within a container of regular or distilled water. It can even be attached to the outside of the container. Over a period of several hours, the pouches are able to transfer detectable KELEA mediated activity into the distilled water. One measure of this acquired activity is very marked and rapid movements, including repulsive behavior, of fine particles of lidocaine that are sprinkled onto the surface of the water. Another measure of KELEA activity is the linear dissolving pattern and passage through the surface of sprinkled particles of neutral red dye. A third measure is the increased volatility of the water as measured by weight loss in closed but not completely sealed containers. Yet another measure is the slight addition to the measured weight of a folded sheet of paper placed adjacent to a container of KELEA activated water. Each of these techniques have been described by the inventor, in the cited publications and the pending patent applications, which as noted above, are included herein by reference. The capacity of paper to withdraw KELEA from water is attributed to its content of branched cellulose molecules. The prior temporary placement of some paper into water is a useful technique to render the water inactive in the described assay procedures. Restoration of these activities using the same can be used to show the subsequent re-acquisition of an increased level of KELEA into water.

The KELEA activated waterceutical pouches are useful for enhancing the ACE pathway in those individuals who are knowledgeable of, and who are willing to wear a pouch. As described in this Application, a method has been developed to more broadly increase the availability of KELEA activated water, which will include those who are not necessarily wearing a waterceutical pouch. The invented method is to humidify the air with KELEA activated water. This has allowed for both human contact of the water vapor via exposed skin and from the inhalation of the KELEA moistened air into the lungs. The concept of using this method arose from the inventor's experience of the added refreshing feelings from including KELEA activated water in room humidifiers. The ability to create environments with increased levels of KELEA is supported by the following observations.

### SUMMARY OF THE INVENTION

In contrast to using KELEA activated water to individually treat individuals with an insufficiency of cellular energy provided by the alternative cellular energy (ACE) pathway, the process of humidification of the environment with KELEA activated water provides a method to deliver KELEA to groups of individuals and, thereby, enhancing the ACE pathways of those present within the humidified environment. This method will greatly increase the access of humans to the disease preventing and disease alleviating ben refits of KELEA activated water.

### DETAILS OF THE INVENTION

Several ultrasonic whole room humidifiers have been used over the preceding several months. These have included a two gallon PureGuardian model H5225WCA (www.guardiantechnologies.com); a one gallon Air-Innovations Model MH-407 (www.air-innovations.tv); a Germquardian Model H2000 (www.germguardian.com).; aHolmes Air Model HM470, a humidifier made by CVS Pharmacy, and others. The water used in the humidifiers have included regular tap water, store purchased distilled water, deionized water (from CalTech), water activated by 30 minutes immersion of the Jing Orb electrolysis device (www.jingorb.com, distributed by BioElectroDynamics Corporation, AZ), Enercel, a homeopathic product also marketed as Benicel provided by World Health Advanced Technologies, Sarasota, Fla., Vortical Energy Water, available from Starchamber Products, Sacramento, Calif.; a naturally activated water available from a deep well at the Bheau View Ranch, San Marcos, Calif., and many water samples directly activated by the inventor. Water has also been indirectly activated using weaterceutical pouches containing the naturally activated water available from the Bheau View Ranch. Before use in a humidifier, the kinetic activity of the activated water can be further increased by repeated jolting (approximately 20-30 times) on a hard surface.

The primary test system has been to use small transparent containers of water, e.g. R1 circular, flat-bottom capped dishes measuring 1 inch diameter and 1 inch in height with the cap, available from The Container Store in Pasadena Calif., and labeled boxbox (item number 10-048399. These dishes are easily examined under an inverted microscope in the lidocaine and neutral red dye assays. The inventor has also used mini-cubes desert-serving containers that can easily hold 50 ml of water. Larger cups filled with water can also be included in the testing.

The main testing comprises using either KELEA-depleted or KELEA-enhanced water in the humidifier. Different sources of water are assessed for their level of KELEA. The environmental level of KELEA activity is assessed using water samples placed into covered small dish containers. The containers are placed into the humidified environments and periodically assessed for their KELEA activity. The initial environments were confined cupboards, but later large whole rooms were humidified. Even for the large rooms, a widespread positive effect was easily achieved with the daily humidification of approximately 250 ml of KELEA activated water.

The KELEA-depleted water for testing in the humidifiers is obtained by leaving a sheet of paper in the water for up to about 30 minutes. After removal, of the paper, the water is tested for the essential lack of any repeated attraction and repulsion of groups of fine lidocaine particles sprinkled onto the water and examined using a low power inverted microscope. Particles of neutral red dye will also remain stationary on the surface of the water as they slowly dissolve into circular red patches. Similar testing is performed on the water in the dishes before and after being placed into the humidified environments.

The results showed that when using KELEA-enhanced water in the humidifier and allowing for sufficient time, the water in the dishes show increased KELEA activity. This is not seen with containers exposed to humidification with KELEA-depleted water. Within a confined cupboard, the indirect activation was seen within minutes. Activation in a larger room required several hours. The effect was seen with the containers with closed lids. Very fine droplets of evaporated water appeared on the inner surface of the lids; which is another indication of water activation being due to the water's increased volatility.

As an incidental finding, when using the whole room exposure to humidified KELEA activated water, it was easier to demonstrate the increase the level of activation when slightly activated water was used in the dishes. This compared to negative results, when KELEA-depleted water in the dishes. Essentially, it is more challenging to initiate the activation of KELEA-depleted water compared to adding activity to existing, partially activated water.

Although not precisely measured, the relative humidity in an area of the room in which a positive result was obtained was estimated to be approximately 80% compared to a beginning humidity in the room of approximately 50%. These estimates were obtained based on the measured reduction in a thermometer reading after the bulb of the thermometer was wrapped in a moistened wick.

While the humidifier is only contributing a portion of the entire water vapor in the room, it is possible that the KELEA activated vapor component is leading to the activation of much of the pre-existing water vapor in the room. If this is so, then more KELEA activated water will be required in a dry (low relative humidity) environment, than in an environment with a higher pre-existing relative humidity. The required amount of KELEA activated water vapor in the various environments can be assessed using lidocaine particles sprinkled onto test dishes of water. For routine use by the public, the movements of sprinkled lidocaine particles on water is a preferable assay for water activation than the dissolving pattern of neutral red dye, which can stain surfaces. It is also easier than measuring the weight loss of closed but not completely sealed containers of the water. The moving and repulsions seen with lidocaine particles can be observed with other components and generally reflect an opposing force to the electrostatic attraction between particles.

Although subjective, there is also a distinguishing positive behavioral change among those residing in a room that is humidified with KELEA activated water. As now repeatedly confirmed, the moisture feels stimulating when sensed on the face and hands. It is more inviting to a desire to take deep breaths. Running the humidifier with KELEA activated water in the bedroom overnight creates a more positive ambiance upon waking, from a more restful sleep than when occurring using the humidifier with KELEA depleted water. Similar to the prolonged duration of activity of the wearable waterceutical pouches, the effects of continually breathing and being otherwise exposed to KELEA activated water vapor will likely have additive benefits over extended periods of time.

It is concluded, therefore, that there are advantages for humidifying indoor room air with KELEA activated water. The same conclusion will apply to humidifying other enclosed spaces, including automobiles, high-occupancy confinement centers, such as prisons, etc. This can also be accomplished using whole building HVAC systems in which the humidity level in both the heated and cooled air flows is typically closely regulated.

It is envisioned that in most circumstances, the humidification of the air with KELEA activated water will be coincidental to the purpose of the individuals being in the space. For example, office buildings, factories, school rooms, hospitals, Situations can also be envisioned in which the humidified environment will be specifically intended to provide for the prevention or cure of medical diseases, such as being created in therapy rooms in health clinics, burn centers, and wellness centers. The inventor is particularly supportive of this type of approach in the prevention and therapy of infectious diseases, including those caused by stealth adapted viruses. Enhancing the ACE pathway will also likely become a major goal in the therapy of mental illnesses. A major advantage of the approach its the capacity to simultaneously benefit groups of patients rather than an individual patient.

These findings are also applicable to other uses of KELEA activated water, such as aeroponics in agriculture, greenhouses, animal facilities, etc. KELEA activated water also has various industrial uses, including the lowering of the specific heat of water in power plants, increasing the efficiency of fuel combustion, etc. Circulating air humidified with KELEA activated water can provide an alternative to the circulation of KELEA activated liquid water in many of these potential applications.

Regarding the water to be used to humidify the environment, there is a clear preference for naturally available KELEA activated water. One such source is the well water from Bheau View Ranch in San Marcos Calif.. Other locations can be easily identified using the KELEA measuring methods described in this and prior patent applications. Pouches of naturally activated water can be used to activate water, with otherwise a low level of KELEA activity. There are also many additional ways to increase the KELEA level in water. These too have been described in prior patent application, which are incorporated herein by reference. Most of the more recent studies have been conducted with the naturally available water from the Bheau View ranch. Another repeatedly tested water is called Benicel, which has no mineral content and allows for the removal of any filtering device from the humidifiers.

In light of the Covid-19 pandemic, it is useful to make a distinction between the current invention and those who are creating an anti-virus aerosol, such as with hypochlorous acid mist. Rather than trying to destroy viruses, the intention of having individuals passively exposed to air humidified with KELEA activated water is to promote the individuals' ACE pathway. This will in turn provide more defense against infection by coronavirus and other pathogens. Humidified KELEA activated water is likely to also improve the proposed KELEA-attracting function of the brain of the exposed individuals. Enhancing the ACE pathway can have many additional medical and psychological benefits. These have been discussed in the cited research publications.

Directly experiencing the benefits of being in a room that is humidified with KELEA activated water, is likely to lead individuals to a better understanding of KELEA and the ACE pathway. This will surely lead to the more direct use of KELEA activated water in wearable pouches. Indeed, providing a pouch to individuals also allows a way to pre-activate the water for use in a humidifier. This advice has been provided to some of the prior recipients of wearable waterceutical pouches. In one example, the client has strapped one of the provided pouches to a container of reverse osmosis water. She drinks the water and also now uses it in a humidifier.

The described approach to creating an environment with increased KELEA compares favorably with the use of expensive medical devices, which presumably are achieving a similar increase in the level of KELEAS. These devices include Lakhovsky's multi-wave oscillators, the Papimi machine of Dr. Panos Pappas, the Energy Enhancement System being marketed by Sandra Rose Michael, etc.. It is also more convenient than visiting highly ionic (and, therefore, KELEA attracting) environments, such as a forest waterfall.

Many other applications of humidifiers containing KELEA activated water are apparent too the inventor and will become better understood by others as they come to understand the importance of the ACE pathway in health and wellness. Predictably, large numbers of individuals will be able to benefit from the described methods.

1. A method to increase the atmospheric level of a beneficial force termed KELEA, an abbreviation for Kinetic Energy Limiting Electrostatic Attraction, in an indoor or other generally enclosed space, by humidifying the space with KELEA activated water.

2. A method for assessing the KELEA level in water by sprinkling a few fine particles of lidocaine onto the surface of the water and observing the movements of the particles, such that when the particles undergo very rapid movements with marked inter-particle repulsions it is indicative of a high level of KELEA activity in the water; in contract to the lidocaine particles remaining essentially stationary and slowly attaching into small groupings of particles, which are indicative of a very low level of KELEA in the water; with intermediate movement patterns of the lidocaine particles being a semi-quantitative, direct measure of the KELEA level in the water,

3. A method for depleting the measurable level of KELEA in a fluid, including water, by placing paper into the water for a sufficient time that the measure of KELEA activity in the water, including the observed movement patterns of sprinkled particles of lidocaine onto the water, is no longer easily observable, after which the paper can be removed from the KELEA-depleted water.

4. The method of claim 2 in which particles of dipolar compounds other than lidocaine, but which behave in a similar manner to lidocaine, are used instead of lidocaine to assess the KELEA activity in water.

5. The method of claim 1 in which individuals can become exposed to and inhale some of the KELEA activated water that is provided by the humidification of the environments of the individuals; this being done as a means of enhancing the alternative cellular energy (ACE) pathway of the individuals, with its accompanying health and wellness benefits.

6. The method of claim 4 in which there are prolonged periods of several hours of the individuals being exposed to an environment with an increased level of KELEA that has resulted from humidifying the air with KELEA activated water.

7. The method of claim 4 in which the benefits of the enhanced ACE pathway are intended to increase the individuals' resistance to infectious diseases, including respiratory viruses and bacteria.

8. The method of claim 4 in which the benefits of the enhanced ACE pathway are intended to increase the individuals' resistance to infectious diseases, including stealth adapted viruses.

9. The method of claim 4 in which the benefits of the enhanced ACE pathway are intended to reduce the level of inflammation in the individuals.

10. The method of claim 4 in which the benefits of the enhanced ACE pathway are intended to improve the higher level functioning of the brain.

11. The method of claim 1 in which the container of the water to be added to the humidifier is first jolted in a manner that increases its KELEA activity as measured by the movements and inter-particle repulsions of particles of lidocaine sprinkled onto the water.

12. The method of claim 10 in which the number of jolts applied to the container of the water to be used in the humidifier is greater than 10.

13. The method of claim 1 in which the water being used has a naturally acquired high level of KELEA activity.

14. The method of claim 1 in which the KELEA activated water does not contain any easily measurable soluble components, including minerals.

15. The method of claim 1 in which a container of KELEA activated water is used to repeatedly activate larger volumes of water, with the larger volumes of secondarily activated water then being used for humidification of an environmental space.

16. The method of claim 1 in which a water-activating device is used to provide the activated water for use in the humidification of the space.

17. The method of claim 1 in which a water-activating compound, including a gas, is used to activate the water for use in the humidification of the space.

18. The method of claim 1 in which the humidification is of the entire air supply provided to a building or large structure through a Heating, Ventilation, and Air Conditioning (HVAC) System

19. The method of claim 1 in which the humidified space is that of a vehicle, including a car, truck, bus, train, or a plane.

20. The method of claim 1, in which the desired level of relative humidity with KELEA activated water is greater than 50%
